(19) United States
(12) Patent Application Publication
(10) Pub. No.: US 2022/0062501 A1
(43) Pub. Date: Mar. 3, 2022

(54) DECELLULARIZED FETAL MATRIX FOR TISSUE REGENERATION

(71) Applicant: Northwestern University, Evanston, IL (US)

(72) Inventors: Robert D. Galiano, Glenview, IL (US); Seok Jong Hong, Northbrook, IL (US); Thomas A. Mustoe, Evanston, IL (US)

(21) Appl. No.: 17/443,590

(22) Filed: Jul. 27, 2021

Related U.S. Application Data
(60) Provisional application No. 63/056,948, filed on Jul. 27, 2020.

(51) Int. Cl. A61L 27/36; A61L 27/38

(52) U.S. Cl. CPC A61L 27/3604; A61L 27/3687; A61L 27/3826; A61L 2430/04; A61L 27/3873; A61L 2430/40; A61L 2430/30; A61L 27/367

(57) ABSTRACT

The present invention provides methods of regenerating muscle tissue and methods of treating volumetric muscle loss comprising administering decellularized fetal matrix scaffold. Also provided are methods for treating soft tissue injury and improving reconstructive surgery.

### CROSS-REFERENCE TO RELATED PATENT APPLICATIONS

The present application claims the benefit of priority under 35 U.S.C. § 119(e) to U.S. Provisional Application No. 63/056,948, filed on Jul. 27, 2020, the content of which is incorporated herein by reference in its entirety.

### STATEMENT REGARDING FEDERALLY SPONSORED RESEARCH

This invention was made with government support under Grant Number W81-XWH-14-2-0004 awarded by the Department of Defense. The government has certain rights in this invention.

### SEQUENCE LISTING

A Sequence Listing accompanies this application and is submitted as an ASCII text file of the sequence listing named “702581_02017_ST25.txt” which is 3400 bytes in size and was created on Nov. 4, 2021. The sequence listing is electronically submitted via EFS-Web with the application and is incorporated herein by reference in its entirety.

### BACKGROUND

Approximately 4.5 million reconstructive surgical procedures are performed annually as a result of car accidents, cancer ablation, or cosmetic procedures. Volumetric muscle loss (VML) is a condition resulting from a variety of causes including traumatic injury, acquired or congenital conditions, or iatrogenic intervention that can cause significant functional impairment and morbidity alongside economic and psychosocial consequences¹. The prevalence of this condition has been rising with the advent of modern military technology and medical care, with wounded warriors now surviving large-scale soft tissue injuries and requiring reconstructive surgery².

VML results from the tendency of skeletal muscle to undergo fibrosis at the site of large defects. Upon loss of a significant volume of muscle, not only are myogenic progenitor cells lost, which cells that are needed for proliferation and differentiation into mature contractile myotubes, but extensive loss of muscle mass also results in depletion of connective tissue and basement membrane, which are essential to ensure proper alignment and structure of the muscle. Without myoblast precursor cells, and without their proper biochemical and biomechanical guidance cues, functional muscle regeneration cannot occur. Experimental data has shown that fibroblast recruitment and collagen deposition effectively outpace migration of myogenic precursor cells, limiting their capacity to enter the zone of injury, differentiate, and bridge the muscular gap, resulting instead in fibrosis and loss of muscle function³. Current treatments for VML involve the use of existing host tissue to construct muscular flaps or grafts. Existing surgical therapies for VML include primary muscle apposition, which is unsuitable for large defects. Muscular autografts and vascularized free muscle transfer can enable partial restoration of function⁴, but strength recovery outcomes are barely satisfactory^{5,6}. These approaches also beget harvest-associated morbidity and vascular compromise of free tissue transfer⁷. Implantation of bioengineered acellular extracellular matrix (ECM) scaffolds has proven promising for VML therapy. Studies have shown functional improvement⁸ and skeletal muscle regeneration⁹ mediated by ECM implantation into muscle defects.

Early gestational fetal cutaneous matrix possesses the remarkable ability to undergo scarless repair without significant fibroplasia or acute inflammation^{10,11}. This intrinsic property of this fetal matrix has been attributed in part to differences in leukocyte and platelet function¹² and properties of fetal fibroblasts¹³, enabling these cells to regenerate organized dermal matrix at the wound site. The characteristics of fetal matrix itself may promote the regenerative process by favoring migration and proliferation in a manner superior to their adult counterparts¹⁴. Hyaluronic acid is more abundant in fetal ECM, is favorably produced under inflammatory conditions in the fetus¹¹, and has been shown to modulate the synthetic activity of fibroblasts¹³. A fine, reticular meshwork of type III collagen with its large pore size allows for ease of cellular migration¹⁵ and forms an integral part of fetal ECM, differentiating it from the dense ropes of type I collagen found in adult matrix. Type III collagen is rapidly redeposited in early-gestation fetal wound beds, a property lost in later gestation and after birth^{16,17}.

As such, there is a need for improved methods of treating volumetric muscle loss and soft tissue injury, include in during reconstructive surgery, which may be met through the use of fetal matrix.

### SUMMARY

The present disclosure provides methods of treating muscle loss and increasing myocyte proliferation and growth in a subject. Further the disclosure provides decellularized fetal matrix and compositions comprising the same.

In one aspect, the disclosure provides a method of treating volumetric muscle loss in a subject in need thereof, the method comprising administering decellularized fetal matrix to the subject in need thereof in an amount sufficient to treat volumetric muscle loss (VML). In some aspects, the decellularized fetal matrix is made by a process comprising: a) obtaining fetal tissue; b) decellularizing the fetal tissue under negative pressure with about 0.25% SDS solution; and c) washing the decellularized fetal tissue to remove residual sodium dodecyl sulfate (SDS) to make decellularized fetal matrix, optionally wherein the decellularized fetal tissue is washed with PBS and a salt solution (e.g. 0.5 mM CaCl₂).

In another aspect, the disclosure provides a method of increasing myocyte proliferation and growth in a subject in need thereof, the method comprising administering decellularized fetal matrix in an amount effective to increase myocyte proliferation and growth in the subject.

In a further aspect, the disclosure provides a decellularized fetal matrix for use in treating volumetric muscle loss or soft tissue injury, the decellularized fetal matrix obtained from xenogeneic or allogenic fetal tissue.

In another aspect, the disclosure provides a composition for muscle regeneration, the composition comprising decellularized fetal matrix.

### BRIEF DESCRIPTION OF THE DRAWINGS

FIG. 1A. Composite soft tissue (top row) harvested from neonatal day 3 rat (left), fetal gestational day 18 rat (middle), and fetal gestational day 24 rabbit (right). H&E staining (bottom row) of composite tissues. Black arrows depict epidermal appendages (hair follicles), blue arrowheads flanking epidermal layer, green arrowheads flanking dermal layer, and red arrowheads flanking panniculus carnosus muscle layer. Scale bars: 200 μm.

FIG. 1B. Scanning electron microscopy (top row) of adult rat decellularized ECM (left column), neonatal ECM (middle column), and fetal ECM (right column). Scale bar: 10 μm. Picrosirius red staining (bottom row). Middle neonatal ECM next to paper seen in lower corner (white). Scale bar: 50 μm.

FIG. 2A. MHC immunofluorescence staining of Day 60-harvested specimen. Central cross-sectional images of defect alone (top left), neonatal rat ECM (top right), fetal rat ECM (bottom left), and fetal rabbit ECM (bottom right). Fixed cells were stained with a MHC-specific antibody and visualized with a fluorescence-labeled secondary antibody (red). Nuclei were counterstained with DAPI (blue). Scale bars: 200 μm.

FIG. 2B. Composite imaging of Day 60 MHC immunofluorescence staining. Cross-sectional tissue of defect alone (top) and fetal rat ECM implant (bottom). Fixed cells were stained with a MHC-specific antibody and visualized with a fluorescence-labeled secondary antibody (red). Nuclei were counterstained with DAPI (blue). Scale bars: 500 μm.

FIG. 2C. ImageJ signal intensity quantification of MHC immunofluorescence staining. Signal intensities were averaged between 4 random images taken within each of the stained defect or matrix tissue samples. Error bars represent mean+standard deviation. *p≤0.05.

FIG. 2D. Western blot for MHC protein expression at the defect site of a day 60 harvested specimen. GAPDH was used as an internal control.

FIG. 3 CD31 immunofluorescence staining of Day 60-harvested specimens. Central cross-sectional images of defect alone (top left), neonatal rat ECM (top right), fetal rat ECM (bottom left), and fetal rabbit ECM (bottom right). Fixed cells were stained with a CD31-specific antibody and visualized with a fluorescence-labeled secondary antibody (green). Scale bars: 100 μm.

FIG. 4A Expression of inflammation-associated genes. mRNA extracted from Day 60-harvested defect and matrix tissues were analyzed using RT-qPCR to quantify expression of pro-inflammatory genes Il1b, Tnf, and Ptgs2. Error bars represent mean+standard error, *p≤0.05, **p≤0.01. n=6 per group.

FIG. 4B. Expression of fibrosis-associated genes. mRNA extracted from Day 60-harvested defect and matrix tissues were analyzed using RT-qPCR to quantify expression of pro-fibrotic genes Ccn2, Col1a1, Tgfb1, and Acta2. Error bars represent mean+standard error, *p≤0.05, **p≤0.01, ***p≤0.001, ****p≤0.0001. n=6 per group.

FIG. 5. Gross appearance of defect alone (top left), neonatal rat ECM (top right), fetal rat ECM (bottom left), and fetal rabbit ECM (bottom right) in the Sprague-Dawley latissimus dorsi defect at Day 60 harvest. Green arrowheads are flanking the residual LD defect. Nylon sutures are visible around the periphery of the defects implanted with matrix. Scale bar: 500 μm.

FIG. 6. Supplemental Table 1. RT-qPCR primer details. Il1b forward SEQ ID NO: 1, reverse SEQ ID NO: 2; Tnfa forward SEQ ID NO: 3, reverse SEQ ID NO: 4; Tgfb1 forward SEQ ID NO: 5, reverse SEQ ID NO: 6; Ptgs2 forward SEQ ID NO: 7, reverse SEQ ID NO: 8; Ccn2 forward SEQ ID NO: 9, reverse SEQ ID NO: 10; Col1a1 forward SEQ ID NO: 11, reverse SEQ ID NO: 12; Acta2 forward SEQ ID NO: 13, reverse SEQ ID NO: 14; Gapdh forward SEQ ID NO: 15, reverse SEQ ID NO: 16.

### DETAILED DESCRIPTION

The present disclosure describes the use of a fetal-derived, acellular soft tissue ECM scaffold (decellularized fetal matrix scaffold) to promote regeneration of skeletal muscle, including in a model of volumetric muscle loss (VML). The inventors compared muscle regeneration in untreated defects to those implanted with both autologous and xenogeneic scaffolds, demonstrating that implantation of decellularized autologous and xenogeneic fetal scaffolds in a VML model resulted in improved myogenesis compared to the outcomes of allowing the defect to heal unaltered. Thus, the present disclosure provides methods of treating volumetric muscle loss, and compositions for acellular and composite therapy for treating soft tissue injury and during reconstructive surgery.

The use of fetal tissue-derived ECM for skeletal muscle recovery is poorly studied despite its ability to undergo scarless wound regeneration compared to adult wound healing. The ECM components of a fetal matrix favor cellular migration and proliferation in a manner superior to its adult counterpart. Hyaluronic acid is more abundant in fetal ECM, is favorably produced under inflammatory conditions in the fetus, and has been shown to modulate the synthetic activity of fetal fibroblasts.

In some embodiments, the present disclosure provides decellularized fetal matrix made by a negative pressure-assisted decellularization protocol and by removing residual sodium dodecyl sulfate (SDS) used in the protocol as described below and in the examples and methods of use. SDS, which is commonly used to extract cells from tissues, causes inflammatory encapsulation and pro-fibrotic fibroblast activation in vivo. The inventors discovered that the ECM composition of fetal matrix is more porous and composed of loosely reticulated collagen fibers in comparison to adult matrix. Advantageously, the fetal matrix allows for substantial myocyte ingrowth using both allogeneic (rat) and xenogeneic (rabbit) fetal matrix-implants when compared to adult matrix implanted groups in a rat latissimus dorsi (LD) muscle defect model. Both allogeneic and xenogeneic fetal ECM demonstrated similar degrees of myogenesis, neovascularization, inflammation, and suppression of fibrosis, making fetal matrix useful for treatment of muscle loss (including volumetric muscle loss), soft tissue injury and reconstructive surgery.

In some embodiments, the present disclosure provides a method of treating volumetric muscle loss in a subject in need thereof, the method comprising: administering decellularized fetal matrix to the subject in need thereof in an amount sufficient to treat volumetric muscle loss.

Volumetric muscle loss (VML) is a condition that results from traumatic injury that can cause significant functional impairment and morbidity along with economic and psychosocial consequences. A prior method for treatment was surgery using muscle autografts, including vascularized muscle flaps and minced muscle grafts, which partially restored function. Porcine small intestinal submucosa (SIS) have also been used. However, these autografts do not lead to regeneration of lost muscle tissue and require donor tissue, resulting in harvest-associated morbidity. Functional outcomes in these patients remain inadequate, largely due to fibrosis.

The present disclosure provides decellularized fetal matrix derived from fetal muscle that retains higher levels of the growth factors, cytokines, and matricryptic peptides necessary for the stimulation of skeletal muscle regeneration, while maintaining a macroscale structure supportive of myogenesis and permissive to vascular ingrowth. As described in the examples, the decellularized fetal matrix comprises extracellular matrix (ECM), growth factors, cytokines and proteins but is devoid of viable cells from the donor (e.g., allogenic or xenogeneic donor). The removal of donor cells reduces the likelihood of an immune reaction to matrix when implanted into a subject in need thereof.

Also described herein, is a method of decellularizing a fetal matrix using a negative pressure decellularization protocol. The term “decellularized fetal matrix” may also be referred to as a “scaffold” and the term could be used interchangeably as the decellularized fetal matrix may be used as a scaffold for which myocytes grow and regenerate and which can also allow for revascularization of the muscle tissue. In some embodiments, the decellularized fetal matrix can be seeded with cells before administering to a subject or can be coadministered with one or more growth factors, immunomodulatory agents or therapeutic agents which enhance the ability to regrow muscles or regain muscle functionality.

Treating of volumetric muscle loss, may include, for example, increasing muscle growth, including new muscle growth (e.g., myocyte growth), retention and/or restoration of muscle function, increased neovascularization of the muscle, and suppression of inflammation and fibrosis within the muscle. VML that can be treated by the methods described herein may result from a variety of causes. For example, VML may be the result of traumatic injury, invasive surgical procedures or congenital or acquired conditions (e.g., musculoskeletal disease) in which regaining muscle mass (e.g., additional muscle growth) and/or function is required.

The present disclosure also provides a method of increasing myocyte proliferation and growth in a subject in need thereof, the method comprising: administering decellularized fetal matrix in an amount effective to increase myocyte proliferation and growth in the subject.

The subject in need of myocyte proliferation and growth may be a subject having soft tissue injury, VML, degenerative muscle disease, or requiring reconstructive surgery, among others. Soft tissue injury (STI) is the damage of muscles, ligaments and tendons through the body. Specifically, soft tissue injuries contemplated are injuries that result in damage and loss of muscle mass. The decellularized fetal matrix may be used to rebuild or regrow a damaged muscle (e.g., a muscle of the face, hand, foot, arm, leg, back or trunk) or soft tissue (e.g., at the interface between an amputated limb and a prosthetic device) or to reconstruct (partially or totally) muscle mass. The decellularized fetal matrix is preferably of a size and volume to provide a therapeutic effect to the subject, for example, to provide sufficient support for implantation in a patient to result in muscle cell growth and vascularization.

In addition, fetal matrices described herein can be used in the reconstruction, including reconstruction of the fine mimetic muscles of face that control facial expression. In another embodiment, the decellularized fetal matrix can be used to reconstruct muscles within the hand and fingers.

In another embodiment, the decellularized fetal matrix described herein can be used to treat hernias. In some embodiments, the decllularized fetal matrix is used to treat abdominal wall hernias.

In some embodiments, the decellularized fetal matrix and compositions comprising the same can be used for reconstructive surgery. Not to be bound by any theory, the decellularized fetal matrix may be used in reconstructive surgery in which muscle mass is missing and/or needed to regain function and structural integrity of the site. In one embodiment, the reconstructive surgery is breast surgery. In another embodiment, the reconstructive surgery is facial surgery. In a further embodiment, the reconstructive surgery is face surgery.

The decellularized fetal matrices described herein may also be used in methods and procedures to reduce of hypertrophic scar formation in methods and procedures to repair injury and wounds. Hypertrophic scare are characterized by deposits of excessive amounts of collagen (deposited from myofibroblasts during the healing process) which gives rise to a raised scar. Hypertrophic scars may formed during wound healing process of an injury.

The methods described herein can use either allogeneic or xenogeneic decellularized fetal matrices. In one embodiment, the decellularized fetal matrix is allogeneic. In another embodiment, the decellularized fetal matrix is xenogeneic. As demonstrated in the examples, both allogenic and xenogeneic decellularized fetal matrix can promote new muscular growth and neovascularization in treatment of volumetric muscle loss (VML) or skeletal muscle injury.

As used herein, “treating” or “treatment” describes the management and care of a subject for the purpose of combating a condition or disorder. Treating includes the administration of the decellularized fetal matrix or composition of present disclosure to prevent the onset of the symptoms or complications, to alleviate the symptoms or complications, or to eliminate the condition or disorder. Treating includes actions for improving the condition of the patient (e.g., the relief of one or more symptoms), delay in the onset or progression of the disease, etc. In some embodiments, treating includes reconstructing skeletal muscle tissue (e.g., where such tissue has been damaged or lost by, e.g., injury or disease) by implanting the decellularized fetal matrix into a subject in need thereof. Treating further includes methods of increasing muscle cell growth or function within a subject in need thereof.

The term “administering” as used herein refers to the implanting or contacting of the decellularized fetal matrix with a subject in need of such treatment. The matrix may be implanted adjacent to the site of injury or need for regrowth of muscle tissue, or in an area that would impart beneficial effects to the site needing muscle growth as would be appreciated by one skilled in the art. Implanting may be carried out by surgical procedures, known by one skilled in the art, to in vivo attach the decellularized fetal matrix to an area in need within the subject. Specifically, the decellularized fetal matrix or compositions disclosed herein can be used to treat a volumetric muscle loss, soft tissue injury or to aid in reconstructive surgery.

As used herein, “subject” or “patient” are used interchangeably and refers to mammals and non-mammals. A “mammal” may be any member of the class Mammalia including, but not limited to, humans, non-human primates (e.g., chimpanzees, other apes, and monkey species), farm animals (e.g., cattle, horses, sheep, goats, and swine), domestic animals (e.g., rabbits, dogs, and cats), or laboratory animals including rodents (e.g., rats, mice, and guinea pigs). Examples of non-mammals include, but are not limited to, birds, and the like. The term “subject” does not denote a particular age or sex. In one specific embodiment, a subject is a mammal, preferably a human. In a preferred embodiment, the human has volumetric muscle loss or muscle injury. In another example, the human has soft tissue injury. In a further example, the human is undergoing reconstructive surgery.

The terms “effective amount” or “therapeutically effective amount” refer to an amount sufficient to effect beneficial or desirable biological or clinical results. That result can be reducing, alleviating, inhibiting or preventing one or more symptoms of a disease or condition, for example, increasing muscle cell growth, muscle regeneration, or muscle function. In some embodiments, the effective amount is an amount suitable to provide the desired effect, e.g., muscle growth, neovascularization or increased muscle function.

The decellularized fetal matrix has several potential advantages over synthetic ECM scaffolds known in the art. First, the decellularized fetal matrix maintains the biochemical and structural complexity of matrix derived from native tissue, which are challenging to mimic with biofabrication techniques alone in synthetic matrices. Second, the decellularized Fetal matrices leads to modulation of inflammatory and fibrotic gene expression (reduced inflammatory and fibrosis at the site of injury) indicative of improved tissue regeneration and decreased tissue fibrosis. Third, the decellularized fetal matrices of the present disclosure are able to promote myocyte proliferation and supporting microvasculature neogenesis in vivo.

In another embodiment, the present disclosure provides a decellularized fetal matrix that comprises ECM and is devoid of donor cells. The decellularized fetal matrix can be made by a process of: a) obtaining fetal tissue; b) decellularizing the fetal tissue under negative pressure in a solution of about 0.2-0.5% SDS solution, preferably about 0.25% SDS; and c) washing the tissue with PBS and 0.5 mM CaCl₂ to remove residual sodium dodecyl sulfate (SDS) to make decellularized fetal matrix. The method can further comprise incubating the decellularized fetal matrix scaffold is treated with DNase I to remove any exogenous DNA. Produced decellularized fetal matrix may be stored in a physiologically acceptable carrier solution until use, for example, in phosphate buffer saline (PBS) at 4° C. The decellularixzed fetal matrix may be xenogeneic to the subject to be treated. In another embodiment, the matrix may be allogeneic.

In some embodiments, the decellularized fetal matrix may be seeded with cells prior to administration. Preferably the cells are allogenic cells to the subject, and seeded prior to administration to the subject, and in some embodiments, the cells are autologous to the subject.

In another embodiment, the decellularized fetal matrix may be co-administered with an additional growth factor, immunomodulatory compound, or therapeutic agent with the decellularized fetal matrix.

In some embodiments, the decellularized fetal matrix may be administered in combination with cells (myocytes or precursor cells), growth factors or compounds, for example, an angiogenic compound, (e.g., vascular endothelial growth factor (VEGF)), immunomodulatory factors (e.g. factors to reduce inflammation), or therapeutic agents which can be seeded on or carried by the decellularized fetal matrix to facilitate the formation of muscles, vascular cells or vasculature in the muscle tissue. Suitable growth factors are known in the art, and include, but are not limited to, for example, angiogenetic compounds, cellular growth factors, among others. Suitable angiogenetic factors are known in the art, and include, for example, VEGF, among others.

Suitable cells for seeding of the matrix are known in the art, and include, for example, myogenic progenitor cells, myocytes, pluripotent stem cells (including induced pluripotent stem cells derived from the subject to be treated) and the like. Muscle cells, preferably skeletal muscle cells (e.g., myocytes), or precursor muscle cells (iPSCs, myocyte precursor cells, etc.) used to seed the decellularized fetal matrix are preferably mammalian muscle cells, including primate muscle cells, preferably human. In some embodiments the cells are precursor cells, or cells that are capable of differentiating into mature, multi-nucleates muscle cells, specifically skeletal muscle cells, under appropriate culture conditions known in the art. Muscle precursor cells are known in the art and can be derived by methods known in the art. See, e.g., U.S. Pat. No. 6,592,623. In some embodiments, the precursor cells are induced pluripotent stem cells derived from the subject to be treated.

“Skeletal muscle cells” include, but are not limited to, myoblasts, satellite cells and myotubes. “Myoblasts” are a type of muscle precursor cell. If the myofiber is injured, the myoblasts are capable of dividing and repopulating it. “Myotubes” are elongated, multinucleated cells, normally formed by the fusion of myoblasts. Myotubes can develop into mature muscle fibers, which have peripherally-located nuclei and myofibrils in their cytoplasm (e.g., as found in mammals). Cells may be syngeneic (i.e., genetically identical or closely related, so as to minimize tissue transplant rejection), allogeneic (i.e., from a non-genetically identical member of the same species) or xenogeneic (i.e., from a member of a different species). Syngeneic cells include those that are autogeneic or autologous (i.e., from the patient to be treated) and isogeneic (i.e., a genetically identical but different subject, e.g., from an identical twin). Cells may be obtained from, e.g., a donor (either living or cadaveric) or derived from an established cell strain or cell line. For example, cells may be harvested from a donor (using standard biopsy techniques known in the art.

A decellularized fetal matrix for use in treating volumetric muscle loss or soft tissue injury is described herein. In a preferred embodiment, the decellularized fetal matrix is obtained from xenogeneic fetal tissue, allowing for the production of larger quantities for treatment of a human subject from non-human sources.

In another embodiment, the disclosure provides a composition for muscle regeneration, the composition comprising decellularize fetal matrix. The decellularized fetal matrix may be stored, prior to administration, in a sterile pharmaceutically acceptable carrier which retains the structure and properties of the matrix.

“Pharmaceutically acceptable” carriers are known in the art and include, but are not limited to, for example, suitable diluents, preservatives, solubilizers, emulsifiers, liposomes, nanoparticles among others. Pharmaceutically acceptable carriers are well known to those skilled in the art and include, but are not limited to, 0.01 to 0.1 M and preferably 0.05M phosphate buffer or 0.9% saline. Additionally, such pharmaceutically acceptable carriers may be aqueous or nonaqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media. In some embodiments, additional components may be added to preserve the structure and function of the matrix of the present disclosure, but are physiologically acceptable for administration to a subject. A thorough discussion of pharmaceutically acceptable excipients is available in REMINGTON'S PHARMACEUTICAL SCIENCES (Mack Pub. Co., N.J. 1991). The compositions used with the present disclosure can be sterilized by conventional, well-known sterilization techniques. The compositions may contain pharmaceutically acceptable additional substances as required to approximate physiological conditions such as a pH adjusting and buffering agent, toxicity adjusting agents, such as, sodium acetate, sodium chloride, potassium chloride, calcium chloride, sodium lactate, and the like.

The composition may be used as an implant for repair of muscle tissue and muscle injury. The term “implant” refers to a product configured to repair, augment or replace (at least a portion of) a natural tissue of a subject (e.g., for veterinary or medical (human) applications). The term “implantable” means the matrix can be inserted, embedded, grafted or otherwise chronically attached or placed on or in a patient. In some embodiments, the implants may include cells seeded thereon and/or comprising growth factors as described herein.

As used herein in the description of the disclosure and the appended claims, the singular forms “a,” “an” and “the” are intended to include the plural forms as well, unless the context clearly indicates otherwise. Furthermore, the terms “about” and “approximately” as used herein when referring to a measurable value such as an amount of a compound, dose, time, temperature, and the like, is meant to encompass variations of +/−20%, 10%, 5%, 1%, 0.5%, or even 0.1% of the specified amount. Also, as used herein, “and/or” or “/” refers to and encompasses any and all possible combinations of one or more of the associated listed items, as well as the lack of combinations when interpreted in the alternative (“or”).

In another embodiment, the decellularized fetal matrix can be used in combination with a therapeutic agent. As used herein, the term “therapeutic agent” refers to any synthetic or naturally occurring biologically active compound or composition of matter which, when administered to a subject, induces a desired pharmacologic, immunogenic, and/or physiologic effect by local and/or systemic action. The term, therefore, encompasses those compounds or chemicals traditionally regarded as drug and biopharmaceuticals including molecules such as proteins, peptides, hormones, nucleic acids, growth factors, gene constructs and the like. Examples of therapeutic agents are described in well-known literature references, such as the Merck Index (14th edition), the Physicians' Desk Reference (64th edition), and The Pharmacological Basis of Therapeutics (12th edition), and they include, without limitation, substances used for the treatment, prevention, diagnosis, cure or mitigation of a disease or illness; substances that affect the structure or function of the body, or pro-drugs, which become biologically active or more active after they have been placed in a physiological environment.

The present invention has been described in terms of one or more preferred embodiments, and it should be appreciated that many equivalents, alternatives, variations, and modifications, aside from those expressly stated, are possible and within the scope of the invention.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

### Illustrative Embodiments

The following embodiments are illustrative and should not be interpreted to limit the scope of the claimed subject matter.

Embodiment 1. A method of treating volumetric muscle loss in a subject in need thereof, the method comprising administering decellularized fetal matrix to the subject in need thereof in an amount sufficient to treat volumetric muscle loss (VML).

Embodiment 2. The method of embodiment 1, wherein the decellularized fetal matrix is xenogeneic.

Embodiment 3. The method of embodiment 1, wherein the decellularized fetal matrix is allogeneic.

Embodiment 4. The method of any one of the preceding embodiments, wherein the decellularized fetal matrix is administered during surgery.

Embodiment 5. The method of embodiment 4, wherein the surgery is reconstructive surgery.

Embodiment 6. The method of any one of the proceeding embodiments, wherein the decellularized fetal matrix is made by a process comprising: a) obtaining fetal tissue; b) decellularizing the fetal tissue under negative pressure with about 0.25% SDS solution; and c) washing the decellularized fetal tissue to remove residual sodium dodecyl sulfate (SDS) to make decellularized fetal matrix, optionally wherein the decellularized fetal tissue is washed with PBS and a salt solution (e.g. 0.5 mM CaCl₂).

Embodiment 7. The method of embodiment 6, wherein the decellularized fetal matrix is treated with DNase I to remove any DNA.

Embodiment 8. The method of any one of the preceding embodiments, the method further comprising seeding the decellularized fetal matrix with allogenic cells prior to administering the decellularized fetal matrix to the subject.

Embodiment 9. The method of embodiment 8, wherein the allogenic cells are myocytes.

Embodiment 10. A method of increasing myocyte proliferation and growth in a subject in need thereof, the method comprising administering decellularized fetal matrix in an amount effective to increase myocyte proliferation and growth in the subject.

Embodiment 11. The method of embodiment 10, wherein the subject has volumetric muscle loss.

Embodiment 12. The method of embodiment 10, wherein the subject suffered soft tissue injury.

Embodiment 13. The method of embodiment 13, wherein the injury is an abdominal wall hernia.

Embodiment 14. The method of embodiment 10, wherein the subject is undergoing reconstructive surgery.

Embodiment 15. The method of embodiment 14, wherein the reconstructive surgery is breast reconstructive surgery.

Embodiment 16. The method of any one of embodiments 10-15, wherein the decellularized fetal matrix scaffold is xenogeneic.

17. The method of any one of embodiments 10-15, wherein the method reduces hypertrophic scar formation.

Embodiment 18. A decellularized fetal matrix for use in treating volumetric muscle loss or soft tissue injury, the decellularized fetal matrix obtained from xenogeneic or allogenic fetal tissue.

Embodiment 19. The decllularized fetal matrix of embodiment 18 further comprising seeded myocytes.

Embodiment 20. A composition for muscle regeneration, the composition comprising decellularized fetal matrix.

### EXAMPLES

The invention will be more fully understood upon consideration of the following non-limiting examples.

### Example 1: Decellularized Fetal Matrix Suppresses Fibrotic Gene Expression and Promotes Myogenesis in a Rat Model of Volumetric Muscle Loss

Traumatic muscle loss often results in poor functional restoration. Skeletal muscle injuries cannot be repaired without substantial fibrosis and loss of muscle function. Given its regenerative properties, we evaluated outcomes of fetal tissue-derived decellularized matrix for skeletal muscle regeneration. We hypothesized that fetal matrix would lead to enhanced myogenesis and suppress inflammation and fibrosis.

Methods: Composite tissue comprised of dermis, subcutaneous tissue, and panniculus carnosus was harvested from the trunk of New Zealand White rabbit fetuses on gestational day 24, from Sprague-Dawley rats on gestational day 18 and neonatal day 3, and decellularized using an SDS-based negative pressure protocol. Six, 10 mm diameter full-thickness rat latissimus dorsi wounds were created for each treatment, matrix implanted (excluding defect groups), and allowed to heal for 60 days. Analyses were performed to characterize myogenesis, neovascularization, inflammation, and fibrosis at harvest.

Results: Significant myocyte ingrowth was visualized in both allogeneic and xenogeneic fetal matrix groups compared to neonatal and defect groups based on MHC immunofluorescence staining. Microvascular networks were appreciated within all implanted matrices. At day 60, expression of Ccn2, Col1a1, and Ptgs2 were decreased in fetal matrix groups compared to defect. Neonatal matrix-implanted wounds failed to show decreased expression of Col1a1 or Ptgs2, and demonstrated increased expression of Tnf, but also demonstrated a significant reduction in Ccn2 expression.

Conclusion: Initial studies of fetal matrices demonstrate promise for muscle regeneration in a rat latissimus dorsi model. Further research is necessary to evaluate fetal matrix for future translational use and better understand its effects.

Materials and Methods

Decellularized Matrix Preparation

Decellularized tissue scaffolds were prepared using a previously described negative pressure-assisted decellularization protocol that preserves matrix structure¹⁸. Briefly, pregnant Sprague-Dawley rats and New Zealand White rabbits were euthanized and fetuses were harvested on gestational days 18 and 24, respectively, as fetal tissue injured in these species at these time points have previously been demonstrated to heal in a regenerative manner^{19,20}. Substantial isolated muscle was unable to be harvested from fetal rats. Thus, composite soft tissues of the back, including dermis, subcutaneous tissue, and panniculus carnosus were harvested from all animal groups. The composite tissue was dissected in one continuous sheet measuring approximately 2×3 cm for fetal rat and 4×5 cm for fetal rabbit tissues. Neonatal rat composite tissue was included as a comparison group because neonatal skin is histologically indistinguishable from that of the adult14. Neonatal rats were euthanized on postpartum day 3 and composite soft tissue was harvested similarly, measuring approximately 4×5 cm. As described previously¹⁸, tissue was decellularized with a 0.25% SDS solution replaced every two hours, incubated with agitation under negative pressure. Tissues were washed in sterile water and SDS was precipitated by washing in a 0.5 mM CaCl₂ solution (Sigma Aldrich). DNA was removed by treatment with DNase I (Sigma Aldrich). Matrices were washed and stored in sterile PBS at 4° C. Before use, matrices were cut to size with 12 mm diameter circular skin biopsy punches (Acuderm) and sterilized for 30 minutes with UV light.

Rat Latissimus Dorsi (LD) Defect Model

All animal experiments were performed according to a protocol approved by the Northwestern University Animal Care and Use Committee. Twelve male Sprague-Dawley rats (Harlan) weighing 300-350 g were anesthetized in an induction chamber using isoflurane and received intraperitoneal injection of 80 mg/kg ketamine with 10 mg/kg xylazine. The animal's dorsum was shaved and prepped using aseptic technique. A 4 cm dorsal midline incision was made through the skin and panniculus carnosus to expose the LD muscles. A 10 mm diameter biopsy punch was used to create a full thickness LD defect bilaterally for a total of 24 defects. Neonatal rat (NRa), fetal rat (FRa), or fetal rabbit (FRb) matrices were placed into the defect and sutured to the surrounding native muscle using 5-0 nylon sutures. Defects without matrix implantation were marked peripherally with nylon sutures. The panniculus carnosus and skin were then closed with a running 3-0 nylon suture. Six wounds per study group were created.

All animals survived surgery and were sacrificed on post-operative day 60. This time point was chosen based on our lab's prior studies evaluating muscle regeneration using commercial matrix vs autologous matrix implantation (paper undergoing revision).

Photographic documentation of the gross appearance of the matrices were obtained at the time of harvest. Implants were excised with a 2 mm border of native muscle outside of suture markings. The disk of tissue was bisected and half was fixed in 10% formalin for 24 hours, then embedded in paraffin for further processing. The remaining half disk of tissue was again bisected and native muscle was excised, leaving only matrix. A quarter of the total matrix was stored in RNAlater (Ambion) and the remaining quarter stored at −80° C. for RNA and protein processing.

Histology and Imaging

Picrosirius red staining was performed on decellularized matrices embedded and frozen in Tissue-Tek OCT (Sakura Finetek) and sectioned to 6 μm. Slides were mounted with Permount (Fisher Scientific) and imaged using the Nikon Eclipse 50i light microscope and the NIS Elements BR software (Nikon).

Scanning electron microscopy (SEM) was performed at the Northwestern University Center for Advanced Microscopy (CAM). Briefly, matrices were fixed with 2% glutaraldehyde in water containing 3% sucrose, washed with water, and dehydrated in serial ethanol.

Samples were transferred to a Samdri-790 critical point dryer (Tousimis) and dried in critical point of CO2. 10 nm gold coating was deposited on samples using Baltec MED 020 sputter coater, and analyzed on the NeoScope benchtop SEM (JEOL) operated at 5 kV using a secondary electron detector.

Tissue specimens were fixed in formalin, embedded in paraffin, and sectioned to 6 μm. Tissues were stained with hematoxylin and eosin (H&E) or, alternatively, immunofluorescent staining was performed using antibodies for CD31 (Santa Cruz Biotechnology 1:500 and myosin heavy chain (MHC, DSHB 1:100), and samples were incubated with fluorophore-labeled secondary antibodies (Invitrogen) and counterstained with 1 μg/mL 4′,6-diamidino-2-phenylindole (DAPI, Santa Cruz Biotechnology). 4 random images were captured at 10× magnification for each of the 6 tissue specimens per study group by a single investigator in an unblinded manner using the EVOS FL cell imaging system (Thermo Fisher Scientific).

Staining was analyzed using ImageJ. Signal intensities were averaged within each of the samples.

Western Blot Analysis

Samples were minced, submerged in RIPA buffer with protease inhibitor, and homogenized using 2.0 mm zirconia beads (Biospec Products) and the MagNa Lyser (Roche). Ten of total extracted protein from each condition were loaded and run on a 6% SDS polyacrylamide gel. Protein was transferred onto a nitrocellulose membrane and incubated with mouse anti-MHC (DSHB 1:5000). The membrane was incubated with horseradish peroxide-conjugated secondary antibody (Vector Laboratories 1:5000). Signal was visualized with the Enhanced Chemiluminescence (ECL) detection kit (GE Healthcare).

Reverse Transcription—Quantitative PCR (RT-qPCR)

Total RNA was prepared by mincing the matrix tissue and placing into Trizol reagent (Sigma Aldrich) with 2.0 mm zirconia beads and homogenized using a MagNa Lyser. Total RNA was extracted following the manufacturer's protocol and DNA was removed using a Turbo DNA-free kit (Ambion). Five μg of RNA was reverse transcribed with Superscript III (Invitrogen) and random primers. Analysis was performed with SYBR green on the ABI StepOnePlus Real Time PCR System (Applied Biosystems) to quantify expression of genes of interest.

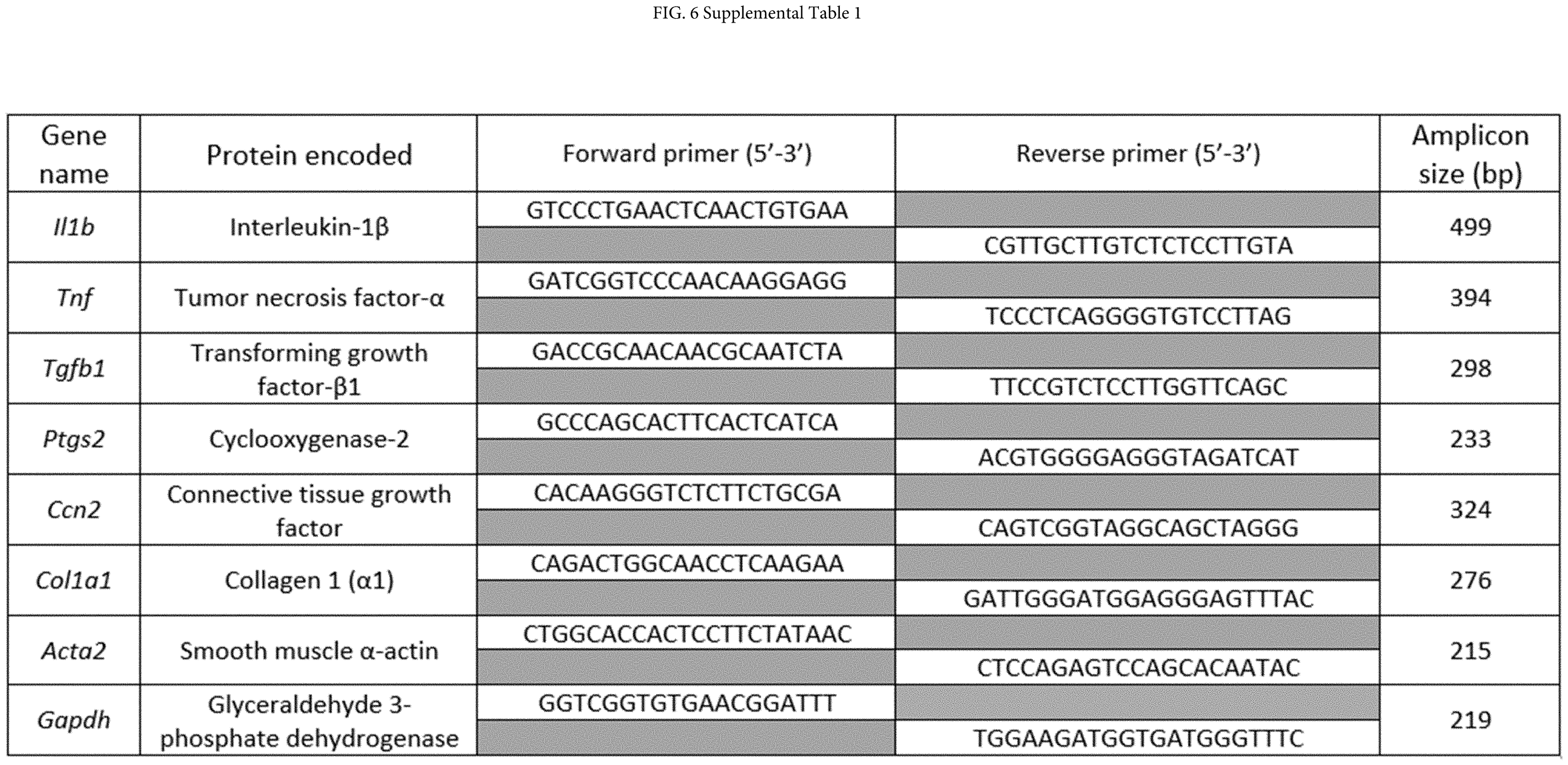

Primer sequences are detailed in FIG. 6 (Supplemental Table 1). Expression of each gene was normalized to Gapdh and fold change was calculated using the 2-ΔΔCT method relative to the defect condition.

Statistical Analysis

Statistical analysis was performed using the GraphPad Prism 8. Results were expressed as mean±standard error. Statistical analysis performed was a one-way ANOVA followed by post-hoc Dunnett's multiple comparisons method of each condition to the defect condition. For all analyses, statistical significance was denoted as p 0.05.

Results

Matrix Composition

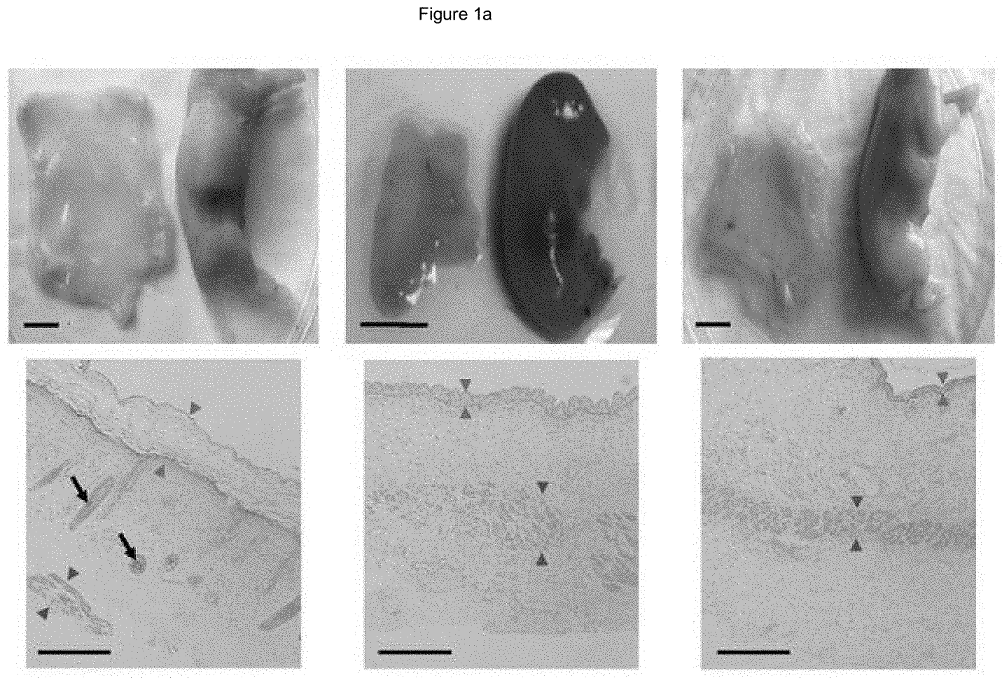

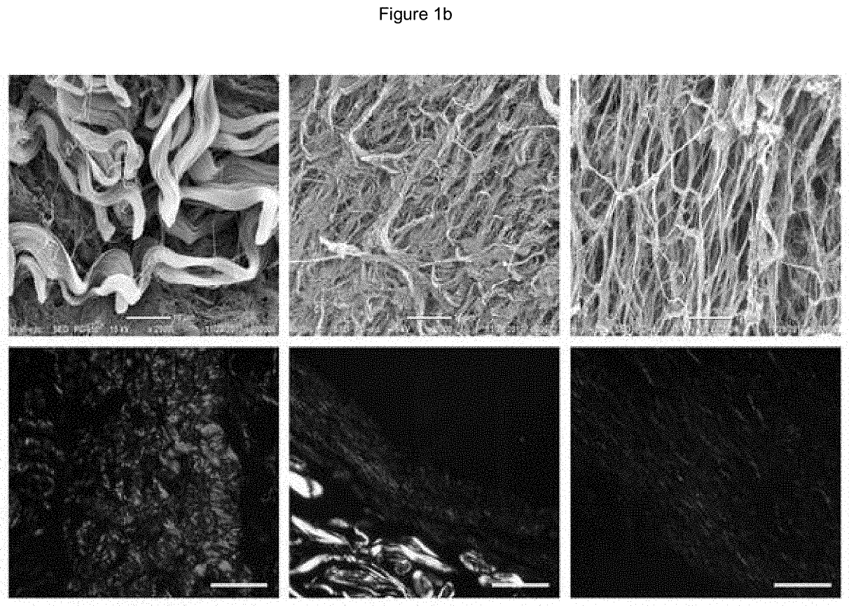

We first wished to investigate the structural differences between neonatal and fetal tissue, and between decellularized matrices derived from these tissues. H&E staining of the harvested composite tissues prior to decellularization demonstrated presence of dermal, subcutaneous, and muscular components. Staining revealed mature, differentiated cells in neonatal composite tissues as evidenced by the presence of epidermal appendages. In comparison, both fetal rat and fetal rabbit composite tissues lacked these structures (FIG. 1a).

Picrosirius red staining, as well as SEM imaging of the decellularized fetal matrices, demonstrated a loosely distributed reticular meshwork of collagen fibers with green birefringence under polarized light. Large pores were visualized between the thin collagen fibers of the fetal matrix. In contrast, decellularized neonatal matrix was comprised of a densely packed network of collagen fibers that birefringed red-orange, similar to that seen in decellularized adult rat soft tissue (FIG. 1b).

Decellularized Fetal Matrix Enhances Myocyte Ingrowth

After characterizing differences between fetal and neonatal matrices, we wished to determine whether these differences in ECM structure yielded different effects on muscle regeneration. At the time of harvest, both autologous and xenogeneic fetal matrices appeared to have integrated well into the surrounding native latissimus dorsi muscle, as they were identifiable only by the non-absorbable nylon sutures and demonstrated gross appearance similar to the surrounding healthy muscle tissue. Neonatal matrix appeared incorporated into the native muscle peripherally, but appeared more attenuated centrally compared to fetal matrix. In comparison, the defects that received no intervention healed through fibrosis with only a thin, translucent scar tissue present at the defect site (FIG. 5).

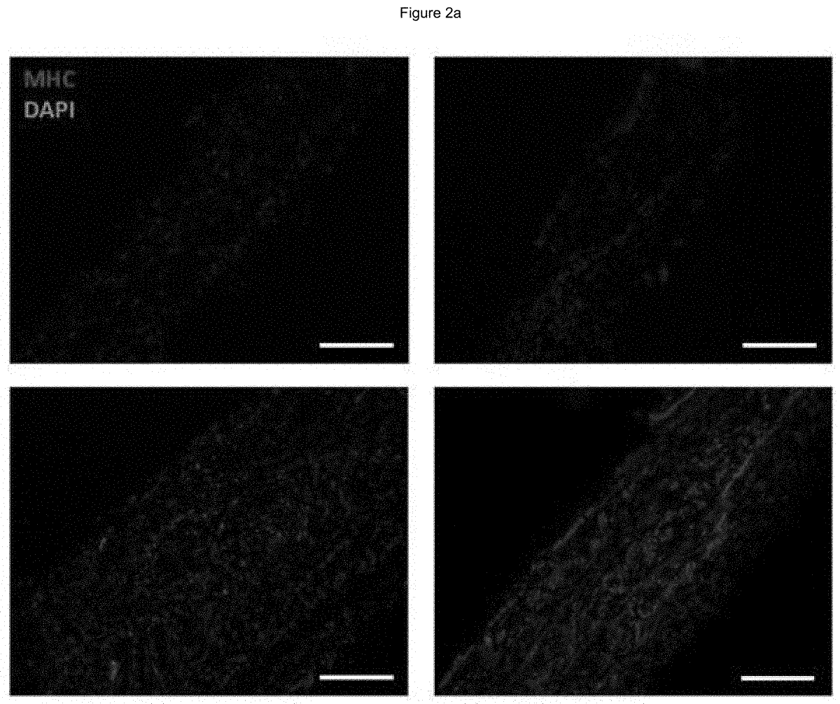

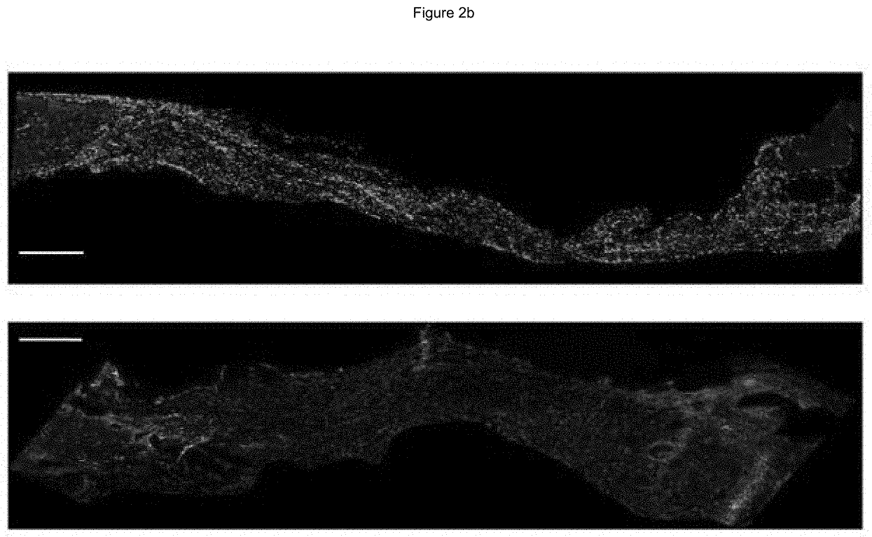

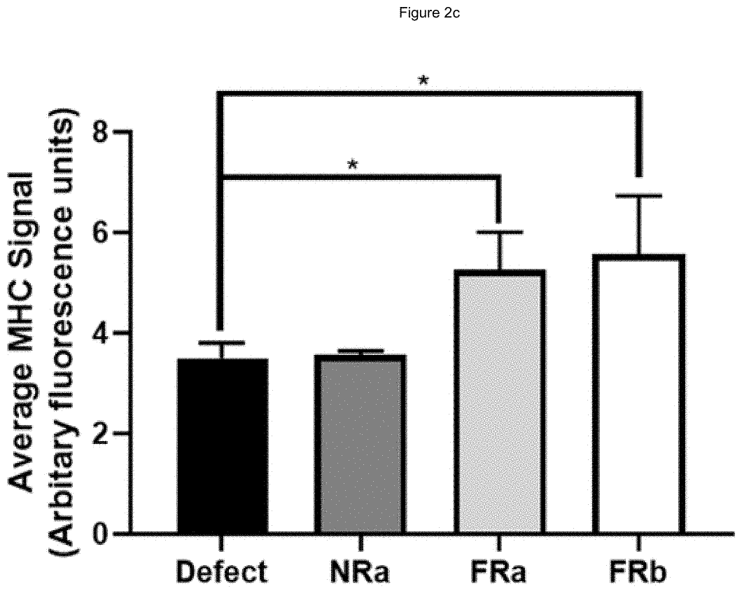

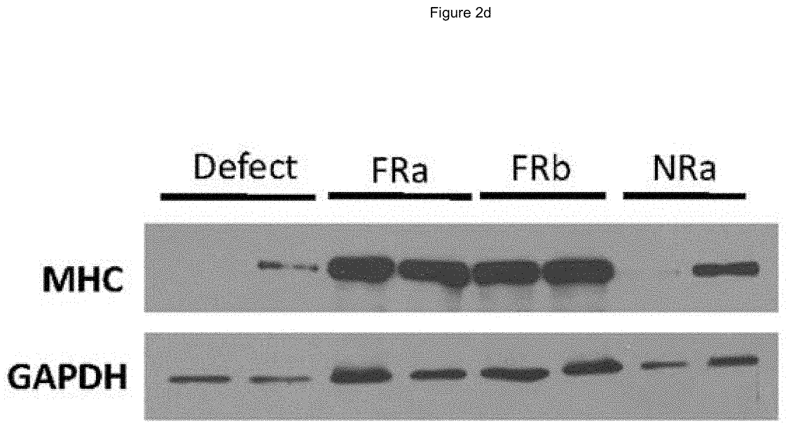

Qualitative analysis by immunofluorescence (IF) staining for MHC revealed minimal myocyte growth centrally within the defect condition and neonatal matrices. Conversely, MHC+ myocytes were distributed throughout both fetal matrices, with myocytes visualized in the most central aspects of the matrix (FIG. 2a,b). Quantification of MHC staining using ImageJ analysis demonstrated that both autologous and xenogeneic fetal matrix implants yielded increased myocyte ingrowth into the defect site compared to defect alone. Neonatal autologous matrix failed to demonstrate a significant increase in myocyte ingrowth (FIG. 2c). Western blot analysis confirmed that both autologous and xenogeneic fetal matrices led to increased MHC expression at the defect site, while neonatal matrix failed to do so (FIG. 2d). Taken together, these data demonstrate that autologous and xenogeneic fetal matrices implanted into an LD defect appeared to enhance myocyte ingrowth as validated by increased MHC expression within fetal matrices, while neonatal autologous matrix failed to promote a similar degree of myogenesis.

Decellularized Matrix Supports Microvascular Ingrowth

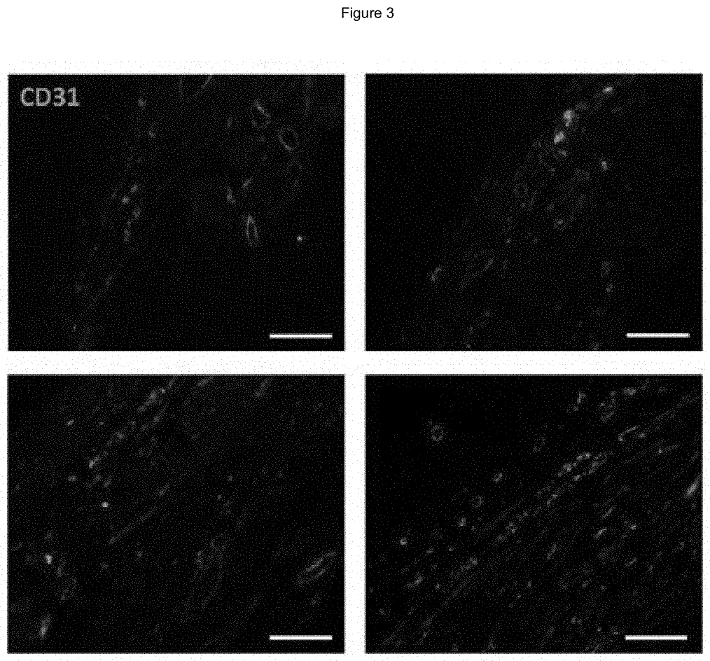

As newly regenerated tissue requires nutritional support, therapeutic utility of ECM for VML treatment requires vascular growth into the matrix. Neovascularization was observed in all study groups sacrificed at day 60 as depicted by vessel-shaped CD31+ staining throughout implanted matrices and non-implanted defect sites (FIG. 3), suggesting that implantation of ECM at the wound site does not prevent neovascularization into newly formed muscle tissue within the matrices.

Fetal Matrix Downregulates Expression of Genes Associated with Inflammation and Fibrosis

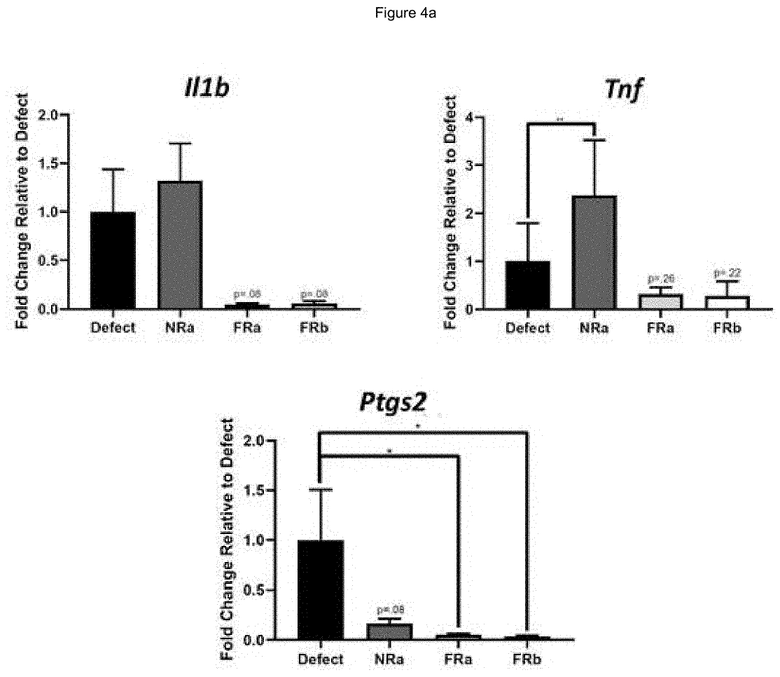

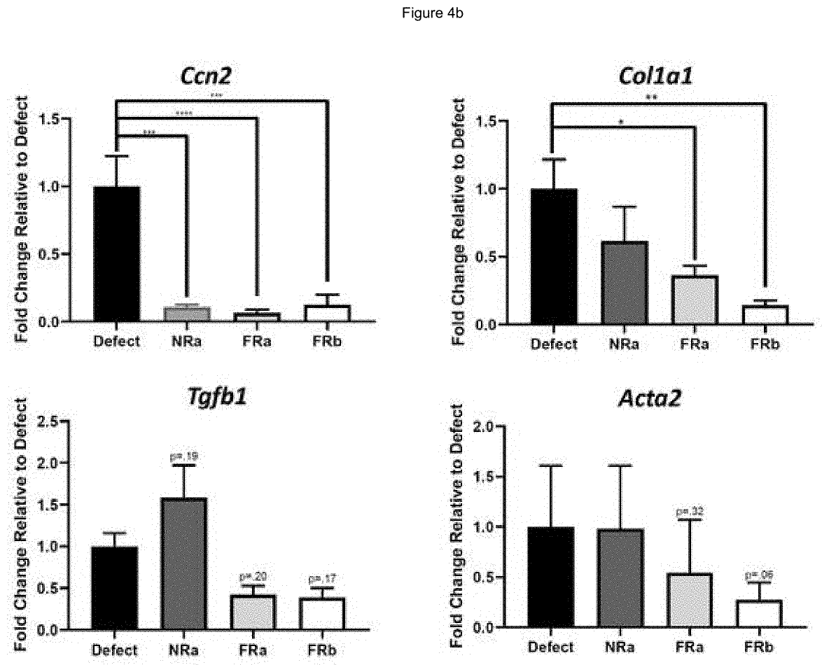

Since fetal and neonatal matrices demonstrated varied propensities for skeletal muscle regeneration, we wished to determine whether matrix implantation into skeletal muscle defects modulated expression of genes associated with inflammation and fibrosis. Tissues from the defect site at day 60 post-operation were harvested and gene expression measured by RT-qPCR. Compared to the defect alone condition, implantation of neonatal rat ECM into the LD defect failed to decrease expression of pro-inflammatory Il1b, while significantly increasing expression of Tnf, the genes encoding IL-1β and TNF-α, respectively. In contrast, implantation of fetal rat and fetal rabbit matrix led to decreased expression of these genes, though these decreases did not reach statistical significance. Expression of Ptgs2, the gene encoding COX2, was decreased under all matrix-implanted conditions, however only reaching statistical significance in the cases of fetal matrices (FIG. 4a).

Quantification of fibrosis-associated gene expression demonstrated that all matrix-implanted conditions showed significantly decreased expression of Ccn2, the gene encoding the pro-fibrotic matricellular protein CTGF, relative to the defect condition. Both fetal matrix-implanted conditions resulted in decreased expression of Col1a1, a gene encoding type I collagen, while neonatal matrix implantation failed to decrease Col1a1 expression significantly. No statistically significant differences were demonstrated between any matrix-implanted condition and the defect alone condition for expression of Tgfb1 and Acta2, the genes encoding the pro-fibrotic growth factor TGF-β1 and myofibroblast contractile protein α-SMA, respectively. Both fetal matrices led to decreased expression of Tgfb1 and Acta2, but these differences did not reach statistical significance (FIG. 4b). Taken together, these data demonstrate that fetal matrix implantation led to decreased expression of several pro-inflammatory and pro-fibrotic genes, suggesting that implantation of fetal matrix results in suppressed inflammatory and fibrotic responses, while implantation of neonatal matrix does not have the same effect consistently.

Discussion

Skeletal muscle provides the ability for our bodies to store nutrients, maintain posture, and support movement. Large volume loss of skeletal muscle remains a condition that lacks effective treatment, and results in severe functional impairment and loss of quality of life. Though implantation of ECM into muscular defects has previously shown promise for functional recovery and regeneration^{8,9}, the use of fetal tissue-derived ECM for skeletal muscle recovery is poorly studied despite interest in scarless fetal wound healing¹⁴.

Decellularized fetal matrix has several potential advantages over synthetic ECM scaffolds, as the biochemical and structural complexity of matrix derived from native tissue is challenging to mimic with biofabrication techniques alone, and as our current understanding of the complexities of muscle extracellular matrix is incomplete.

As we were unable to isolate adequate muscle tissue for the LD defect from either the fetal or neonatal rat specimen, we thus chose to use composite soft tissue that included a muscular component in the form of the panniculus carnosus. While porcine cutaneous tissues are more similar to those of humans, we chose to use fetal rabbit as our xenogeneic model given its anatomic similarities to the rat. Both animals used in this experiment contain a panniculus carnosus muscle layer lacking in human and porcine tissues, thus avoiding the additional variable of comparing decellularized composite tissue to either dermal or muscle scaffold alone. Additionally, studies have demonstrated the biocompatibility of implanting New Zealand White rabbit trachea in Sprague-Dawley rats, eliciting minimal immune response^{21,22}. For these reasons, we chose to limit the scope of this study to compare the effects of composite fetal and neonatal tissues in autologous rat and xenogeneic rabbit models.

Here, we demonstrate that the ECM composition of fetal rat and rabbit matrix is more porous and composed of loosely reticulated collagen fibers, compared to neonatal and adult rat matrices (FIG. 1). While fetal skin is associated with scarless wound healing, neonatal skin more closely resembles adult skin histologically, both of which undergo fibrosis in response to injury²³. Thus, we hypothesized that fetal matrix implantation would result in enhanced muscle ingrowth within a VML defect compared to neonatal matrix or native healing of the defect.

Fetal matrix integrated well into surrounding native muscle in the rat LD defect model as assessed by gross appearance at time of harvest (FIG. 5). Fetal matrix promoted myocyte ingrowth into the defect site, as assessed by immunofluorescent (FIG. 2a-c) and Western blot (FIG. 2d) detection of MHC within the fetal matrix after harvest, whereas neonatal matrix failed to promote myocyte ingrowth. Since implanted matrices were acellular, improved muscle growth observed in fetal matrix-implanted samples may be attributed to its structural composition and biochemical cues that modulate the regenerative potential of the surrounding tissues.

All study groups demonstrated neovasculogenesis as observed by the presence of tubular CD31 staining patterns within the defect site (FIG. 3). Microvascular ingrowth was not quantified, as deficiencies in angiogenesis are not characteristic of the inflammatory or fibrotic response, concordant with our observation of neovascularization within the VML defect group. The inflammatory response is associated with the production of proangiogenic mediators that may in fact lead to excessive angiogenesis, contributing to pathological fibrosis²⁴. We were not interested in the angiogenic capacity amongst the treatment groups during this study, but rather to demonstrate the ability for the implanted matrices to support neovascularization necessary for myogenesis.

Recently, a prolonged inflammatory response has been described following VML in a rodent model²⁵, complete with upregulation of inflammation-associated genes and monocyte infiltration at the wound site, ultimately leading to fibrosis. The same group also demonstrated prolonged inflammation following VML in a swine model²⁶. Thus, we investigated whether matrix at the defect site alters inflammatory and fibrotic gene expression. Relative to defect tissue alone, implantation of fetal matrices led to significant downregulation of the gene encoding COX2, as well as nonsignificant decreases in the genes encoding IL-1β and TNF-α. In contrast, neonatal matrix implantation failed to significantly decrease expression of the genes encoding COX2 and IL-1β, and led to a significant increase in expression of the gene encoding TNF-α. Thus, fetal matrices, but not neonatal matrices, appear to suppress expression of several pro-inflammatory genes in a VML model. Similarly, fetal matrix implantation led to significantly decreased expression of genes encoding type I collagen and CTGF, two canonical fibrosis-associated proteins, and nonsignificant decreases in expression of the genes encoding α-SMA and TGF-β1. In contrast, neonatal matrix failed to significantly decrease expression of the genes encoding type I collagen, α-SMA, or TGF-β1, but did decrease expression of the gene encoding CTGF. Taken together, these data suggest that implantation of fetal matrices leads to modulation of inflammatory and fibrotic gene expression indicative of improved tissue regeneration and decreased tissue fibrosis.

As there is a paucity of research looking at the use of fetal matrix for muscle regeneration, our pilot study was meant to demonstrate proof of concept. We acknowledge multiple limitations to this study including small sample sizes, predominantly qualitative analyses, absence of blinding, and lack of functional analysis of de novo regenerated muscle.

However, we believe that our results demonstrate an encouraging degree of internal consistency among parameters measured in autologous and xenogeneic fetal matrices, prompting further studies more adequately powered to measure other quantitative experimental endpoints including matrix composition and functional analysis. While there remain concerns regarding recellularization of thicker matrices for larger muscle defects, we believe that decellularized fetal matrix has the potential to play a therapeutic role for reconstruction of the fine mimetic muscle of facial expression, which can significantly affect patient quality of life. Furthermore, we recognize that it would be ethically impractical to use human fetal tissues for clinical purposes, and thus we have chosen to study xenogeneic fetal tissue matrix. Although this model using fetal rabbit tissue limits translational applicability to humans, our findings encouragingly demonstrate comparable inflammatory, fibrotic, and myogenic responses between autologous and xenogeneic fetal tissues. Moving forward, we are transitioning to larger animal models with the use of porcine matrices for evaluation of tissue-specific scaffold effects on muscle regeneration and functional recovery. As there are various decellularized porcine tissue products already available for reconstructive purposes, we believe these future studies will benefit the translation of our research to clinical practice.

The Example demonstrates that decellularized fetal matrix is a viable acellular therapy, or a component of composite therapy, to promote muscular regeneration. Both autologous and xenogeneic fetal ECM demonstrated similar myogenesis, neovascularization, and suppression of inflammatory and fibrotic gene expression, suggesting potential translational use. The fetal matrix may be used for tissue-specific matrix, or to incorporate cells into ECM scaffolds.

### REFERENCES

1. Garg, K., et al. Volumetric muscle loss: persistent functional deficits beyond frank loss of tissue. Journal of orthopaedic research 33, 40-46 (2015).
2. Bilmes, L. Soldiers returning from Iraq and Afghanistan: The long-term costs of providing veterans medical care and disability benefits. KSG Working Paper No. RWP07-001 (2007).
3. Larouche, J., Greising, S. M., Corona, B. T., & Aguilar, C. A. Robust inflammatory and fibrotic signaling following volumetric muscle loss: a barrier to muscle regeneration. Cell death & disease 9(3), 409 (2018).
4. Walley, K. C., Taylor, E. M., Anderson, M., Lozano-Calderon, S. & Iorio, M. L. Reconstruction of quadriceps function with composite free tissue transfers following sarcoma resection. Journal of surgical oncology 115, 878-882 (2017).
5. Pritsch, T., Malawer, M. M., Wu, C. C., Squires, M. H. & Bickels, J. Functional reconstruction of the extensor mechanism following massive tumor resections from the anterior compartment of the thigh. Plastic and reconstructive surgery 120, 960-969 (2007).
6. Fan, C., et al. Functional reconstruction of traumatic loss of flexors in forearm with gastrocnemius myocutaneous flap transfer. Microsurgery 28, 71-75 (2008).
7. Coulet, B., Boch, C., Boretto, J., Lazerges, C. & Chammas, M. Free Gracilis muscle transfer to restore elbow flexion in brachial plexus injuries. Orthopaedics & traumatology: surgery & research 97, 785-792 (2011).
8. Aurora, A., Roe, J. L., Corona, B. T. & Walters, T. J. An acellular biologic scaffold does not regenerate appreciable de novo muscle tissue in rat models of volumetric muscle loss injury. Biomaterials 67, 393-407 (2015).
9. Valentin, J. E., Turner, N. J., Gilbert, T. W. & Badylak, S. F. Functional skeletal muscle formation with a biologic scaffold. Biomaterials 31, 7475-7484 (2010).
10. Chen, W., Grant, M. E., Schor, A. & Schor, S. Differences between adult and foetal fibroblasts in the regulation of hyaluronate synthesis: correlation with migratory activity. Journal of cell science 94, 577-584 (1989).
11. Kennedy, C. I., Diegelmann, R. F., Haynes, J. H. & Yager, D. R. Proinflammatory cytokines differentially regulate hyaluronan synthase isoforms in fetal and adult fibroblasts. Journal of pediatric surgery 35, 874-879 (2000).
12. Olutoye, O. O., Yager, D. R., Cohen, I. K. & Diegelmann, R. F. Lower cytokine release by fetal porcine platelets: a possible explanation for reduced inflammation after fetal wounding. Journal of Pediatric Surgery 31, 91-95 (1996).
13. Mast, B. A., Diegelmann, R. F., Krummel, T. M. & Cohen, I. K. Hyaluronic acid modulates proliferation, collagen and protein synthesis of cultured fetal fibroblasts. Matrix 13, 441-446 (1993).
14. Larson, B. J., Longaker, M. T. & Lorenz, H. P. Scarless fetal wound healing: a basic science review. Plastic and reconstructive surgery 126, 1172-1180 (2010).
15. Murphy, C. M., Haugh, M. G., O'Brien F. J. The effect of mean pore size on cell attachment, proliferation and migration in collagen-glycosaminoglycan scaffolds for bone tissue engineering. Biomaterials 3, 461-466 (2010).
16. Whitby, D. & Ferguson, M. The extracellular matrix of lip wounds in fetal, neonatal and adult mice. Development 112, 651-668 (1991).
17. Beanes, S. R., et al. Confocal microscopic analysis of scarless repair in the fetal rat: defining the transition. Plastic and reconstructive surgery 109, 160-170 (2002).
18. Friedrich, E. E., et al. Residual sodium dodecyl sulfate in decellularized muscle matrices leads to fibroblast activation in vitro and foreign body response in vivo. Journal of tissue engineering and regenerative medicine 12, e1704-e1715 (2018).
19. Robinson, B. W. and Goss, A. N. Intra-uterine healing of fetal rat cheek wounds. Cleft Palate Journal 18, 251-255 (1981).
20. Krummel, T. M., et al. Fetal response to injury in the rabbit. Journal of Pediatric Surgery 22, 640-644 (1987).
21. Sun, F., et al. Structural integrity, immunogenicity and biomechanical evaluation of rabbit decellularized tracheal matrix. Journal of Biomedical Materials Research 103, 1509-1519 (2015).
22. Lange, P., et al. Characterization of a biologically derived rabbit tracheal scaffold. Journal of Biomedical Materials Research 105, 2126-2135 (2017).
23. Coolen, N. A., Schouten, K. C., Middelkoop, E. & Ulrich, M. M. W. Comparison between human fetal and adult skin. Archives of dermatological research 302, 47-55 (2010).
24. DiPietro, L. A. Angiogenesis and wound repair: when enough is enough. Journal of Leukocyte Biology 100, 979-984 (2016).
25. Aguilar, C. A., et al. Multiscale analysis of a regenerative therapy for treatment of volumetric muscle loss injury. Cell Death Discovery 4, 33 (2018).
26. Greising, S. M., et al. Unwavering Pathobiology of volumetric muscle loss injury. Scientific Reports 7, 13179 (2017).

What is claimed:

1. A method of treating volumetric muscle loss in a subject in need thereof, the method comprising administering decellularized fetal matrix to the subject in need thereof in an amount sufficient to treat volumetric muscle loss (VML).

2. The method of claim 1, wherein the decellularized fetal matrix is xenogeneic.

3. The method of claim 1, wherein the decellularized fetal matrix is allogeneic.

4. The method of claim 1, wherein the decellularized fetal matrix is administered during surgery.

5. The method of claim 4, wherein the surgery is reconstructive surgery.

6. The method of claim 1, wherein the decellularized fetal matrix is made by a process comprising:
a) obtaining fetal tissue;
b) decellularizing the fetal tissue under negative pressure with about 0.25% SDS solution; and
c) washing the decellularized fetal tissue to remove residual sodium dodecyl sulfate (SDS) to make decellularized fetal matrix, optionally wherein the decellularized fetal tissue is washed with PBS and a salt solution (e.g. 0.5 mM CaCl₂).

7. The method of claim 6, wherein the decellularized fetal matrix is treated with DNase I to remove any DNA.

8. The method of claim 1, the method further comprising seeding the decellularized fetal matrix with allogenic cells prior to administering the decellularized fetal matrix to the subject.

9. The method of claim 8, wherein the allogenic cells are myocytes.

10. A method of increasing myocyte proliferation and growth in a subject in need thereof, the method comprising administering decellularized fetal matrix in an amount effective to increase myocyte proliferation and growth in the subject.

11. The method of claim 10, wherein the subject has volumetric muscle loss.

12. The method of claim 10, wherein the subject suffered soft tissue injury.

13. The method of claim 13, wherein the injury is an abdominal wall hernia.

14. The method of claim 10, wherein the subject is undergoing reconstructive surgery.

15. The method of claim 14, wherein the reconstructive surgery is breast reconstructive surgery.

16. The method of claim 1, wherein the decellularized fetal matrix scaffold is xenogeneic.

17. The method of claim 1, wherein the method reduces hypertrophic scar formation.

18. A decellularized fetal matrix for use in treating volumetric muscle loss or soft tissue injury, the decellularized fetal matrix obtained from xenogeneic or allogenic fetal tissue.

19. The decllularized fetal matrix of claim 18 further comprising seeded myocytes.

20. A composition for muscle regeneration, the composition comprising decellularized fetal matrix.


SEQUENCE LISTING (16 sequences)

SEQ ID NO: 1 - DNA, 21 bp, Artificial Sequence
  Synthetic- Il1b F
gtccctgaactcaactgtgaa

SEQ ID NO: 2 - DNA, 21 bp, Artificial Sequence
  Synthetic- Il1b R
cgttgcttgtctctccttgta

SEQ ID NO: 3 - DNA, 20 bp, Artificial Sequence
  Synthetic- Tnfa F
gatcggtcccaacaaggagg

SEQ ID NO: 4 - DNA, 20 bp, Artificial Sequence
  Synthetic- Tnfa R
tccctcaggggtgtccttag

SEQ ID NO: 5 - DNA, 20 bp, Artificial Sequence
  Synthetic- Tgfb F
gaccgcaacaacgcaatcta

SEQ ID NO: 6 - DNA, 20 bp, Artificial Sequence
  Synthetic- Tgfb R
ttccgtctccttggttcagc

SEQ ID NO: 7 - DNA, 20 bp, Artificial Sequence
  Synthetic- Ptgs2 F
gcccagcacttcactcatca

SEQ ID NO: 8 - DNA, 20 bp, Artificial Sequence
  Synthetic- Ptgs2 R
acgtggggagggtagatcat

SEQ ID NO: 9 - DNA, 20 bp, Artificial Sequence
  Synthetic- Ccn2 F
cacaagggtctcttctgcga

SEQ ID NO: 10 - DNA, 20 bp, Artificial Sequence
  Synthetic- Ccn2 R
cagtcggtaggcagctaggg

SEQ ID NO: 11 - DNA, 20 bp, Artificial Sequence
  Synthetic- Col1a1 F
cagactggcaacctcaagaa

SEQ ID NO: 12 - DNA, 22 bp, Artificial Sequence
  Synthetic- Col1a1 R
gattgggatggagggagtttac

SEQ ID NO: 13 - DNA, 22 bp, Artificial Sequence
  Synthetic- Acta2 F
ctggcaccactccttctataac

SEQ ID NO: 14 - DNA, 21 bp, Artificial Sequence
  Synthetic- Acta2 R
ctccagagtccagcacaatac

SEQ ID NO: 15 - DNA, 19 bp, Artificial Sequence
  Synthetic- Gapdh F
ggtcggtgtgaacggattt

SEQ ID NO: 16 - DNA, 21 bp, Artificial Sequence
  Synthetic- Gapdh R
tggaagatggtgatgggtttc